SEQUENCE LISTING (2 sequences)

SEQ ID NO: 1 - DNA, 28 bp, Artificial Sequence
  Primer
ttgtcatttgtggccagcacggagctga

SEQ ID NO: 2 - DNA, 27 bp, Artificial Sequence
  Primer
gtcctgaatctaccaacagggttcgac

(19) United States
(12) Patent Application Publication
(10) Pub. No.: US 2013/0266933 A1
(43) Pub. Date: Oct. 10, 2013

(54) PROCEDURE FOR DETERMINING VARIANTS OF INFECTIOUS PANCREATIC NECROSIS VIRUS IN AQUATIC ANIMALS; ASSOCIATED DETECTION KIT; AND USE OF THE PROCEDURE IN AQUATIC ANIMALS

(71) Applicants: David Farcas Guendelman, Cerrillos (CL); Jaime Tobar Rubio, Cerrillos (CL); Sofia Jerez Ortega, Cerrillos (CL); Mario Caruffo Madrid, Cerrillos (CL)

(72) Inventors: David Farcas Guendelman, Cerrillos (CL); Jaime Tobar Rubio, Cerrillos (CL); Sofia Jerez Ortega, Cerrillos (CL); Mario Caruffo Madrid, Cerrillos (CL)

(73) Assignee: CENTRO VETERINARIO Y AGRICOLA LIMITADA, Cerrillos, Santiago (CL)

(21) Appl. No.: 13/992,887

(22) PCT Filed: Dec. 9, 2011
(86) PCT No.: PCT/IB11/55593

§ 371 Date: Jun. 10, 2013

(30) Foreign Application Priority Data
Dec. 10, 2010 (CL) 1400-2010

(51) Int. Cl. C12Q 1/70

(52) U.S. Cl. CPC C12Q 1/701
USPC 435/5

(57) ABSTRACT

The present invention is related to a low cost, fast, specific, sensitive, and suitable for routine application in monitoring activities, procedure for determining variants of infectious pancreatic necrosis virus (IPNV) on samples of different origin; and associated kit.

The present invention is related to a procedure for determining variants of infectious pancreatic necrosis virus (IPNV) for the control of viral infections in aquatic animals, with special attention to fish, and more specifically a procedure for determining the IPNV variants in a mixture, for example, a vaccine, a cell culture or a field sample. The invention comprises the kit and an operational procedure for detection of IPNV, important in the development of international aquaculture industry. The invention presents great sensibility and specificity, is of easy and quick application and presents great efficiency.

### BACKGROUND OF THE INVENTION

### Increasing Importance of Aquaculture in Food Supply Worldwide

With the constant increase of population worldwide, the food supply around the globe from the aquaculture sector has increased. This represents a challenge for the sector, and therefore it is urgent to address the need of improvement of productivity of aquaculture industry worldwide. One of the important factors to achieve sustainable development in the sector is disease management. Diseases in fish are caused from different factors, and diseases caused by virus are the ones that are particularly important for aquaculture industry worldwide.

### Importance of Disease Handling in Diseases Caused by Virus in Aquaculture

Infectious diseases constantly threat sustainability of aquaculture industry, being the ones with a viral origin of the most difficult management. For example, diseases caused by the pancreas disease virus (PDV), infectious salmon anemia virus (ISAV) and infectious pancreatic necrosis virus (IPDV) cause huge losses in the aquaculture industry in Chile as well as in the world.

### Importance of the Infectious Pancreatic Necrosis Virus in Aquaculture

The infectious pancreatic necrosis was first reported in a Brook trout hatchery in 1941 (McGonigle, Trans. Am. Fish. Soc. 70: 297, 1941) and in 1960, the viral nature of the disease was confirmed (Wolf et al, Proc. Soc. Exp. Biol. Med. 104: 105-110, 1960).

Infectious pancreatic necrosis virus (IPNV) is the causative agent of infectious pancreatic necrosis (IPN), a highly contagious and destructive disease, producing necrosis and inflammation of pancreas, and affecting a diverse group of hosts in the whole world, such as rainbow trout, Brook trout, Atlantic salmon, carps, mollusks, and crustaceans (Pilcher and Fryer, Crit. Rev. Microbiol. 7: 287-364, 1980; Wolf, Fish viruses and fish viral diseases, 1988; Hill and Way, Annu. Rev. Fish Dis. 5, 55-77, 1995). Clinical signs in diseases fish include bulging abdomen, erratic swimming, dark pigmentation of skin, and local necrotic injuries in exocrine pancreatic tissue, and under examination of the interior of an affected fish, a white mucus is observed in pathognomic stomach of the disease (McKnight and Roberts, Br. Vet. J. 132:76-86, 1976). Young fish, from two to four months old, seem to be more susceptible to an infection of IPNV, resulting in high mortality (Wolf et al, U.S. Dept. Int. Bur. Sport Fish and Wildlife; Fish Disease Leaflet 1:14, 1966; Frantsi and Savan, J. Wildlife Dis. 7:249-255, 1971). In case of trouts, IPNV attacks younger fish around five to six weeks after their first feeding.

IPNV can cause strong outbreaks in production systems, in which virulent strains of IPNV can cause a mortality higher than 90% in fish younger than four months, and can cause a severe delay in the growth of fish surviving the infection, which remain as asymptomatic carriers/hosts during their whole life, acting as infection reservoirs and spreading IPNV in the medium (Mangunwiryo and Aguis, J. Fish Dis. 11, 125-132, 1988; Reno et al, J. Fish. Res. Board Can. 35, 145-1456, 1978; McAllister et al, Dis. Aquat. Org. 2: 235-237, 1987; McKnight and Roberts, Br. Ven. J. 132: 76-86, 1976; Billi and Wolf, J. Fish. Res. Bd. Can. 26:1459-1465, 1969; Yamamoto, Can. J. Micro. 21:1343-1347, 1975; Hedrick, Ph.D. Thesis, “Persistent Infections of Salmonid Cell Lines With Infectious Pancreatic Necrosis Virus: A Model for the Carrier State in Trout,” Oregon State University, 1980).

Therefore, diseased fish as well as those asymptomatic IPNV carrier fish represent a serious problem for the aquaculture industry, since the only method for control currently available for virus elimination in carrier/host fish is the elimination of those fish. Consequently, IPNV virus is a pathogenic agent of great economic importance in aquaculture industry in Chile and the world.

### Classification of IPNV

IPNV belongs to Aquabirnavirus genus from Birnaviridae family (Dobos, Ann. Rev. Fish Dis. 5: 24-54, 1995; Van Regenmortel et al, Virus Taxonomy, VIIth Report of the ICTV: 481-486, 2000).

### Classification of IPNV Serogroups and Genogroups

Aquabirnavirus, such as IPNV, can be classified in two serogroups, called A and B, based on the results of crossed neutralization with antibodies (Hill and Way, Annu. Rev. Fish Dis. 5, 55-77, 1995). Serogroup A has 9 serotypes, including the majority of IPNV isolates associated with disease in salmonids, and serogrpup B with only one serotype (Hill and Way, Annu. Rev. Fish Dis. 5, 55-77, 1995).

Apart from traditional serological classification, a recent classification of Aquabirnavirus at a genetic level exists, based on the phylogenetic analysis of the main capsid protein from the virus (VP2) (Blake et al, Dis. Aquat. Org. 45, 89-102, 2001; Cutrín et al, Applied and Environmental Microbiology 70, 1059-1067, 2004; Zhang and Suzuki, Journal of Fish Diseases 27, 633-643, 2004; Nishizawa et al, Journal of General Virology 86, 1973-1978, 2005; Bain et al, Journal of Fish Diseases, 31, 37-47, 2008).

### IPNV Virion

IPNV virion measures approximately 60 nm in diameter, does not have an envelope and presents a single icosaedrical capsid (Dobos, Nucl. Acids Res. 3:1903-1919, 1976; Dobos, J. Virol. 21:242-258, 1977; Fauquet et al, Virus Taxonomy: Classification and Nomenclature of Viruses: Eighth Report of the International Committee on the Taxonomy of Viruses, 561-569, 2005). The main structural proteins in the virion are classified as VP1 (4% of the total mass of the virion), VP2 (62%), VP3 (28%) and VP3a (6%) (Dobos, Annu. Rev. Fish Dis. 5:24-54, 1995).

### IPNV Genome

IPNV is the prototype virus for Birnaviridae family, a double stranded, bisegmented RNA genome family (Dobos, Nucl. Acids Res. 3:1903-1919, 1976; Dobos, J. Virol. 21:242-258, 1977; Van Regenmortel et al, Virus Taxonomy, VIIth Report of the ICTV: 481-486, 2000). The smallest genomic segment, called segment B of 2784 bp, is monocystronic and codifies a 94 kDa protein called VP1, which is the putative RNA dependent RNA polymerase (FIG. 1B) (Duncan et al, Virology 181, 541-552, 1991; Duncan et al, J. Virol. 61, 3655-3664, 1987; Dobos, Virology 208: 19-25, 1995). The long genomic segment, called segment A of 3097 bp, is bicystronic. An large open reading frame exists in this segment codifying for a 107 kDa poly-protein (NH₂-preVP2-VP4-VP3-COOH) which is cleaved in a co-traductional way for the protease activity of protein VP4 (27.5 kDa), producing the capside proteins VP2 (54 kDa) and VP3 (31 kDa) (FIG. 1A) (Chang et al, Can. J. Microbiol. 24:19-27, 1978; Huang et al, J. Virol. 60:1002-1011, 1986; Dobos, J. Virol. 21: 242-258, 1977; Duncan et al, J. Virol. 61: 3655-3664, 1987). The poly-protein presents cleavage sites for the protease between aminoacids 508 and 509 in the joint of VP2 and VP4 and between aminoacids 734 and 735 of the joint between VP4 and VP3 (Dobos, J. Virol. 21:242-258, 1977; Duncan et al, J. Virol. 61, 3655-3664, 1987; Petit et al, J. Virol. 74:2057-2066, 2000). The other open reading frame present in segment A precedes and partially overlaps that of the first poly-protein and codifies a non-structural, arginin-rich 15 kDa protein called VP5 (FIG. 1A). Although this protein is not present in the virion, it is detected in infected cells (Heppell et al, J. Gen. Virol. 76, 2091-2096, 1995; Magyar and Dobos, Virology 204: 580-589, 1994).

### Virulence Determinants of IPNV and VP2 Capsid Protein

IPNV genome presents a great variation when compared to other viruses (Heppell et al, Virology 214, 40-49, 1995) and presents different degrees of virulence in isolates from the same serotype (Bruslind and Reno, J. Aquat. Anim. Health 12, 301-315, 2000; Shivappa et al, Dis. Aquat Org. 61:23-32, 2003).

The gene codifying VP2, main protein component of external viral capsid, has two hypervariable short segments in the middle of the codifying region. Changes of nucleotides in these variable genetic regions correlate with differences in the virulence of IPNV observed in controlled conditions and in virulent and avirulent isolates present in wild fish. At a protein level, VP2 presents the majority of epitopes of neutralizing antibodies. (Frost et al, J. Gen. Virol. 76, 1165-1172, 1995; Tarrab et al, J. Gen. Virol. 76, 551-558, 1995; Heppell et al, Virology 214, 40-49, 1995). In this one, the more variable residues correspond to positions 217, 221, 247 and 500 (Blake et al, Dis. Aquat. Org. 45, 89-102, 2001), from which the positions 217 and 221 are responsible for differences in virulence of strains of IPNV of the same serotype, specially the residue in position 221. Highly virulent isolates present threonine residues at position 217 and alanine in position 221, and instead, avirulent isolates present threonine in position 221 (Bruslind and Reno, J. Aquat. Anim. Health 12, 301-315, 2000; Santi et al, Virology 322:31-40, 2003; Shivappa et al, Dis. Aquat Org. 61:23-32, 2003; Santi et al, Virology 322:31-40, 2004; Song et al, J. Virology, August: 10289-10299, 2005). Changing aminoacid in position 221 from threonine to alanine is correlated to the change of the virulent phenotype to attenuated in field isolates maintained in culture in CHSE-214 cells and the same attenuation and change in aminoacid occurs in carrier fish (Munro et al, J. Fish Dis., 29, 43-48, 2006).

### Current Diagnostic Methods for IPNV Infection

Since IPNV can originate strong outbreaks in trout and salmonid production systems, causing high morbidity and mortality in cultures and consequently, great economic losses in aquaculture sector, it is necessary a fast and large scale monitoring to reduce the probability of outbreaks. For this reason, different methods have been developed for detecting IPNV virus (Winton, Ann Rev Fish Dis: 83-93, 1991). These methods include isolating virus from candidate fish in established CHSE-214 cell lines and confirm the identity of the virus. This is done by serum neutralization, ELISA assays, in situ hybridization assays using biotinylated primers, immunogold labeling assays and immunohistochemistry and conventional or real time RT-PCR assays. Among these methods, real time RT-PCR is the fastest and most sensitive method for detection.

### IPNV Variant Analysis

All the previously described methods are useful in diagnostic of IPNV, but none of them is able to establish the nature of IPNV variant found in a sample.

Currently, the IPNV variant analysis is made through cDNA or RT-PCR product sequencing, obtaining from the extracted RNA from diverse sources of samples, with the objective of obtaining the nucleotide sequence and deducing the VP2 amino acid sequence.

Variant analysis through sequencing of VP2 gene zones present operational disadvantages for use as a routine analysis in vaccine or diagnostic control. Said method is of a high cost and demands long times for obtaining results. Furthermore, sequencing can produce ambiguous results depending on the size of the sample.

The procedure proposed herein is the first in allow diagnostic and variant analysis in the same kit. A RT-PCR and further restriction analysis is a low cost, fast, specific, and with good sensitivity analysis, that can be applied in routine form for the control of immunological products or diagnostic of IPNV, and mainly in determining the IPNV variants present. Allows monitoring in vaccine development plants and presents great utility in validation of the strain used for viral propagation in cell cultures, and due to the low cost of implementation, allows routine use in aquaculture industry.

### GENERAL DESCRIPTION OF THE INVENTION

The present invention is related to a low cost, fast, specific, sensitive, and of routine application for monitoring procedure for determining IPN virus variants of samples of different origins. Said procedure contemplates the infection of CHSE-214 confluent cells with IPN virus isolates and further culture thereof. Then, RNA is extracted from infected cells and is used in a reverse transcription reaction for synthesis of cDNA which will be used as template in amplification of a fragment of VP2 protein. The fragment is purified and cloned in a vector which is further used in transformation of chemo competent cells. The colonies presenting the interest insert, which are positive for a VP2 fragment in amplification using PCR, are cultured in liquid medium and subjected to purification of plasmid DNA. Finally, the DNA fragment amplified from VP2 is subjected to a restriction analysis for evaluation of a sequence coding for aminoacids in positions 217 and 221 of protein VP2.

Accordingly, an object of the present invention is providing a procedure for determining virus variants of infectious pancreatic necrosis in vaccines, cell cultures and/or isolates field samples, comprising the necessary components to perform the procedure.

### DETAILED DESCRIPTION OF THE INVENTION

The present invention describes a procedure for determining variants of infectious pancreatic necrosis virus in samples of different origin, such as vaccines, cell cultures and field samples. The procedure comprises the following steps:

A. VIRUS PROPAGATION. Chinook salmon embryo 214 cells (CHSE-214) are prepared until reaching 100% confluence. These cells are cultivated at 18° C. in plastic bottles having 25 cm² surface in MEM (Gibco) medium, supplemented with 0.08% sodium bicarbonate, 0.238% HEPES, 1% fungizone (amphotericin B) (HyClone) and 10% fetal bovine serum (HyClone). Cells are cultivated in plastic bottles having 25 cm² surface.

Confluent cells are infected with seed vials of IPN virus of virulent and avirulent strains. 1 vial for each 25 cm2 of culture of CHSE-214. The culture is maintained at 18° C. until reaching 100% cytopathic effect, which is reached in 5-6 days.

B. RNA EXTRACTION. 600 μl supernatant from the cell culture are taken and 600 μl TRIzol (Invitrogen) or TRI Reagent (Ambion) are added. Agitation for 10 seconds. Afterwards, 200 μl chloroform are added and agitation for 20 seconds. 10 minutes incubation at room temperature. Centrifugation at 12,000 g for 15 minutes at 4° C. and the supernatant is recovered. 800 μl Isopropanol are added and it is let to precipitate for 15 minutes at room temperature. Centrifugation at 16,000 g for 15 minutes at 4° C. The supernatant is discarded and the precipitate is washed with 600 μl 70% ethanol, prepared with nuclease-free water. The precipitate is let to dry for no more than 20 minutes and is resuspended in 50 μl nuclease-free water.

C. RT-PCR. A reverse transcription is performed using Improm-II (Promega) kit according to the following protocol: a mix in 0.2 ml tubes of 2 μl viral RNA, 2 μl nuclease-free water and 1 μl Random Primers (Promega) 4 μg/μl. Incubation at 70° C. for 5 minutes and cooled down at 4° C. for 5 minutes. The reaction is paused and 4 μl RT buffer 5×, 2.4 μl MgCl₂ 25 mM, 1 μl dNTP's 10 μM (Promega), 1 μl RT, 1 μl RNAse inhibitor (RNasin®, Promega), 6.1 μl nuclease free water are added. Then, the program: 5 minutes at 25° C., 1 hour at 45° C., 15 minutes at 70° C. is followed.

Afterwards, the PCR program for amplification of a VP2 fragment is performed. To a 0.2 ml tube 4 μl viral template cDNA, 25 μl GoTaq Green Master Mix (Promega), 3 μl oligo VirVp2-R (Sequence: 5′-TTGTCATTTGTGGCCAGCACGGAGCTGA-3′), 2.3 μl oligo VirVp2-F (Sequence: 5′-GTCCTGAATCTACCAACAGGGTTCGAC-3′) 16 μl nuclease free water are added. The following PCR program is used: initial denaturation: 3 minutes at 94° C., 35 cycles of: 30 seconds at 94° C., 30 seconds at 56° C., 30 seconds at 72° C. and a final extension for 5 minutes at 72° C.

PURIFICATION AND CLONING. The amplified product is checked using visualization of the amplificate in an agarose gel. Afterwards, a DNA purification is prepared from the gel, loading the full volume in a 1% agarose gel. The gel is run for 35 minutes at 90 volts. A purification is performed from the gel using SV Gel Clean-Up System kit (Promega) eluting in a final volume of 30 μl.

The amplified fragment is inserted in pGem-T Easy (Promega) vector following the protocol suggested by the manufacturer. Chemo competent E. coli JM110 cells are prepared, using the calcium chloride method. The cells are transformed with the pGem-T Easy vector containing the cloned fragment from VP2 from either the virulent or avirulent strain. The cells are sow in LB agar plates and incubated at 37° C. for 15-18 hours or until obtaining colonies.

COLONY CHECKING. A selection of transformed E. coli colonies is made and are inoculated in 5 ml LB medium (1% Triptone, 0.5% NaCl, 0.5% yeast extract, 100 μg/ml ampicillin) incubation for 20 hrs. approximately, a PCR is performed for checking the insert used in the protocol and program previously described and as template, 2 μl of culture broth are used.

The colonies presenting the insert are subjected to plasmid DNA extraction using Miniprep (Axygen) kit and are further sent for sequencing.

Each of the plasmids sent for sequencing are subjected to PCR for amplification of the inserted fragment using the program previously described and then the PCR product is subjected to digestion with restriction enzymes.

D. RESTRICTION ASSAY.

Two restriction assays are performed, for checking the two corresponding sites to amino acids 217 and 221 of VP2. A direct digestion using 10 μl of PCR product and the enzymes Mae III (Roche) for amino acid 217 and BanII (New England Biolabs) or its isoschizomer Eco24 (Fermentas) for amino acid 221. The digestion is performed according to the protocols described by the manufacturer for each enzyme and the result is checked in a high resolution 5% agarose gel at 70 volts for 1 hr.

### DESCRIPTION OF THE FIGURES

FIG. 1. Schematic representation of RNA genome of IPNV, segments A and V and its proteins.

FIG. 2. Flow diagram showing the steps of the procedure according to the invention for determining IPNV variants.

FIG. 3. Restriction analysis of two vaccines. Digestion of a PCR product amplified from cDNA from vaccines SRS-IPN SI-22260901 and quad CU-22290901. In one case a greater amount of IPNV SpThr (lane 2, mixed vaccine), is observed, and in the other a greater apparent amount of IPNV SpAla (lane 3, quad vaccine). The first lane shows the molecular weight marker in base pairs (bp).

FIG. 4. Restriction analysis from supernatant of a cell culture infected with virulent IPN obtained from ADL Laboratories, passaged in CHSE-214 5 times (5th passage). Digestion generates only one fragment of 365 base pairs, which indicates that the IPN virus present in the cell culture is attenuated. The samples were amplified with random and specific primers. The first lane shows the molecular weight marker in base pairs (bp).

FIG. 5. Restriction analysis from aliquote from vial IPN PM-24619 IPNv Sp virulent from ADL Laboratories. Digestion generated two fragments (in the image only one is appreciated since both have similar sizes) of around 200 base pairs, which indicates that the IPN virus present in the vial is virulent. The cDNA samples used for template for VirVP2 fragment synthesis were amplified with random and specific primers. The first and last lane show the molecular weight marker in base pairs (bp).

FIG. 6. Analysis of IPN variants in fish isolates. CM and VQ correspond to fish isolated from different hatcheries. SP: Piscicultura; +R: restriction enzyme digestion control; C_{A}+ alanin digestion control; C_{T}+: threonine digestion control. Digestion shows the presence of both IPN variants, virulent and attenuated, since two sizes of bands are present, one of 365 base pairs and the other of approximately 200 base pairs. The first and last lane show the molecular weight standard (std) in base pairs (bp).

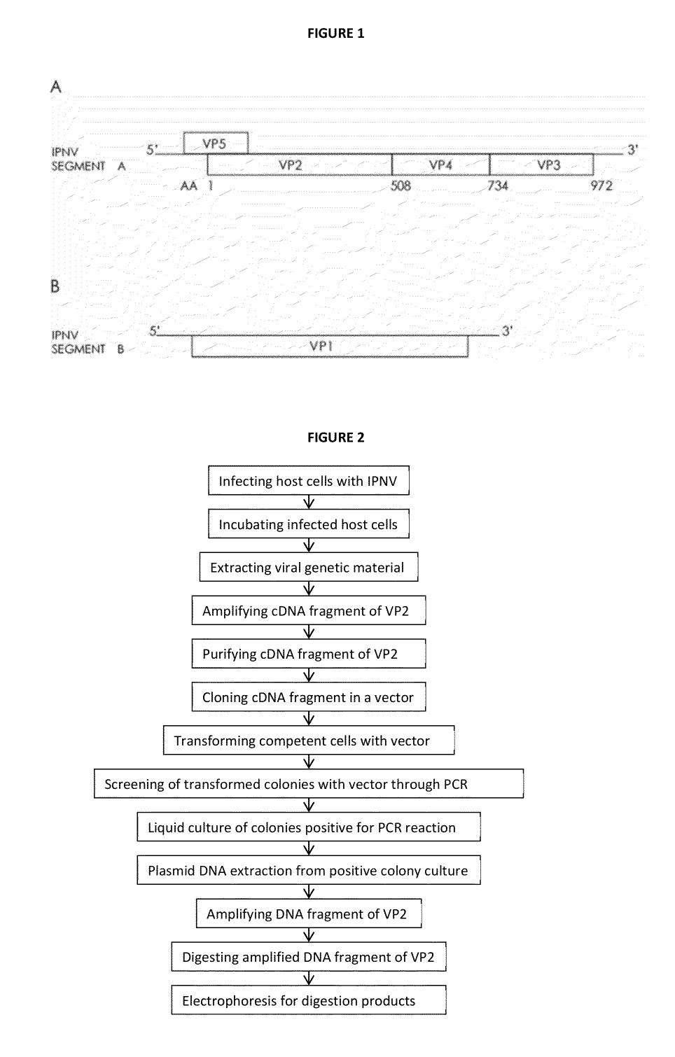

### APPLICATION EXAMPLES

### Example 1

### RFLP for Validation of Attenuated and Virulent IPNv Strains in Vaccine Preparations

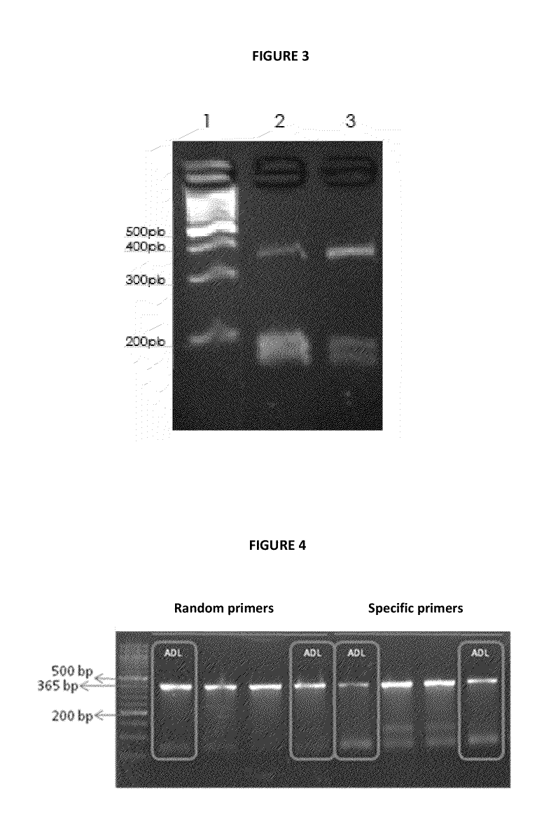

Restriction Fragment Length Polymorphism (RFLP) was used for evaluation of presence of attenuated and virulent strains in a preparation of a commercial vaccine. Direct RNA extraction was performed from the immunological product corresponding to two inactivated injectable vaccines (virina) against IPNV in monovalent formulations (SRS-IPN SI-22260901), as well as polyvalent (quad CU-22290901s) commercially available at Centrovet Ltd. Afterwards, a DNA fragment sequence coding for VP2 protein was amplified, which presents molecular determinants of virulence, through a RT-PCR reaction. Consequently, the cDNA fragment was amplified for a key region in VP2 which was subjected to restriction enzyme digestion as previously indicated (1 enzyme unit per 1 μg DNA for 60 minutes at 37° C.), the digestion product was further analyzed in a 3% agarose gel using BrEt. The migration pattern was characteristic depending on the virus strain, either virulent (alanine) or avirulent (threonine), as observed in FIG. 3.

### Examples 2, 3 and 4

Similarly to the conditions of Example 1, the procedure of the present invention was applied to samples from a cell culture, a vial, and a fish tissue sample. Details on these samples of different origin are summarized in Table 1.

TABLE 1

| Example | Sample | Record |
|---|---|---|
| 2 | Cell culture | CHSE-214 infected with virulent IPN |
| | | virus passaged in CHSE-214 cell |
| | | culture, obtained from ADL (box |
| | | showing lanes in gel of FIG. 4) |
| 3 | Vial | IPN PM-24619 IPNv Sp virulent isolate |
| | | from ADL Diagnostic Chile Ltda. ® |
| | | (box showing lanes in gel FIG. 5) |
| 4 | Fish tissue sample | Fish samples obtained in field |
| | | outbreak |

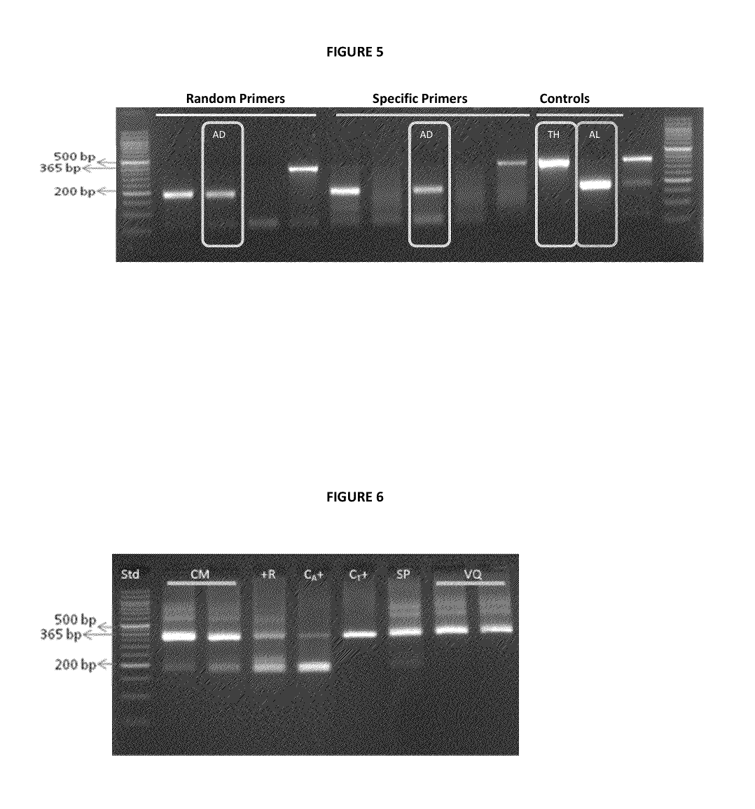

The procedure has been shown to be sensitive and efficient in detecting IPNV variants from samples of different origin. As observed from the restriction/digestion analysis in FIGS. 3 to 6.

1. A procedure for detecting infectious pancreatic necrosis virus (IPNV), comprising the following steps: infecting host cells with virus isolates, incubating said host cells in a culture medium, extracting viral genetic material from host cells, amplifying a cDNA fragment, purifying the amplified cDNA fragment, cloning said cDNA fragment in a vector, transforming competent cells with said vector comprising the desired insert, screening transformed colonies with the vector for positive PCR amplification of the desired insert, liquid culture of select colonies positive for PCR reaction of the desired insert, extracting plasmid DNA from the colonies positive for PCR reaction culture, amplifying the desired DNA fragment through PCR, digesting the amplified DNA with restriction enzymes, and subjecting to electrophoresis the products of the digestion.

2. The procedure according to claim 1, wherein the host cells are derived from cell cultures that can sustain replication of IPNV virus, including Chinook salmon embryo cells (CHSE-214), salmon head kidney (SHK-1) cells, Atlantic salmon kidney (ASK-1) cells or cells from Epithelioma papulosum cyprini (EPC).

3. The procedure according to claim 1, wherein the virus isolates are obtained from vaccines, field isolates and/or cell cultures.

4. The procedure according to claim 1, wherein the amplification of a virus fragment is performed using VirVP2-F and VirVP2-R primers.

5. The procedure according to claim 1, wherein VirVP2-R primer has the following sequence:
(SEQ ID NO 1)
5′-TTGTCATTTGTGGCCAGCACGGAGCTGA-3′.

6. The procedure according to claim 4, wherein VirVP2-F primer has the following sequence:
(SEQ ID NO 2)
5′-GTCCTGAATCTACCAACAGGGTTCGAC-3′.

7. The procedure according to claim 1, wherein the amplified cDNA or DNA fragment is derived from different strains of the infectious pancreatic necrosis virus.

8. The procedure according to claim 7, wherein virulent and avirulent strains are included.

9. The procedure according to claim 1, wherein in the digestion of the amplified DNA fragment is used a specific restriction enzyme cutting the nucleotide sequence corresponding to position 217 of protein VP2.

10. The procedure according to claim 1, wherein in the digestion of amplified DNA fragment a specific restriction enzyme cutting the nucleotide sequence corresponding to position 221 of protein VP2.

11. The procedure according to claim 9, wherein restriction enzyme MaeIII or isoschizomers thereof is used.

12. The procedure according to claim 10, wherein restriction enzymes BanII, Eco24 or isoschizomers thereof is used.

13. The procedure according to claim 1, for diagnosing the presence of infectious pancreatic necrosis virus, and determining variants thereof in aquatic animals.

14. The procedure according to claim 13, wherein the aquatic animals are fish, mollusks or crustaceans.

15. Kit for detecting infectious pancreatic necrosis virus, comprising elements required for performing the procedure according to claim 1.
